(19) United States
(12) Patent Application Publication
(10) Pub. No.: US 2024/0384322 A1
(43) Pub. Date: Nov. 21, 2024

(54) Process Challenge Device and Method

(71) Applicant: 3M INNOVATIVE PROPERTIES COMPANY, St. Paul, MN (US)

(72) Inventors: G. Marco Bommarito, Stillwater, MN (US); Joshua D. Erickson, Blaine, MN (US); Feng Zeng, Shanghai (CN)

(21) Appl. No.: 18/564,946

(22) PCT Filed: May 18, 2022
(86) PCT No.: PCT/IB2022/054634

§ 371 Date: Nov. 28, 2023

(30) Foreign Application Priority Data
Jun. 4, 2021 (CN) 202110626008.9

(51) Int. Cl. C12Q 1/22; C12M 1/12; C12Q 1/04; C12Q 1/18

(52) U.S. Cl. CPC C12Q 1/22; C12M 37/06; C12Q 1/04; C12Q 1/18

(57) ABSTRACT

The disclosed process challenge device is a casing and a self-contained biological indicator operatively connected to the casing. The biological indicator has a housing with an open end and an interior space, a plurality of viable test microorganisms or an active enzyme in the housing, a sterilant pathway extending through the housing to the test microorganisms or the active enzyme, a detection reagent in the interior space, and an aqueous liquid in an openable container. The aqueous liquid is in selective communication with the viable test microorganisms or active enzyme. A first portion of the biological indicator has an open end and is disposed in the casing and a second portion of the self-contained biological indicator is disposed outside the casing. The second portion of the biological indicator comprises a part of the housing through which the aqueous liquid and detection reagent can be observed.

### TECHNICAL FIELD

This disclosure relates generally to process challenge devices, in particular to process challenge devices using process indicators such as biological indicator organisms or biological enzymes disposed in a self-contained biological indicator, used to assess the efficacy of procedures for the inactivation of microorganisms in industry is related to health care, food packaging and preparation, and other industries that use biological indicators.

### BACKGROUND

A variety of products and articles, including medical instruments, must be sterilized prior to use to prevent contamination of a sample, a wound site, or the like with a microorganism (e.g., a pathogenic microorganism) present on or in the product or article. A number of sterilization processes are used which involve contacting the product or article with a fluid sterilant, such as a gaseous sterilant. Examples of such sterilants include, for example, steam, ethylene oxide, hydrogen peroxide, ozone, and the like.

The products and articles are generally packaged such that the sterilant can pass through the packaging, but microorganisms cannot pass through. Even though the sterilant can pass therethrough, the packaging restricts the movement of the sterilant to (and/or into) the product or article. Moreover, some products and articles include spaces within them that can only be reached by the sterilant via a restricted path. For example, endoscopes often include a long, narrow channel through which the sterilant must pass in order to sterilize the interior surfaces of the endoscope. These and other forms of restrictions associated with products and articles to be sterilized must be considered when employing a particular sterilization process, so that all surfaces of the product or article are exposed to the sterilant for a time sufficient to cause sterilization.

Monitoring for sufficient sterilization is generally carried out by placing an appropriate sterilization indicator along with the product and/or article to be sterilized within a sterilization chamber. A variety of sterilization indicators, including biological and chemical indicators, are known, and used for this purpose. However, to emulate the above described restrictions embodied in the various products and articles, the sterilization indicator can be placed in a process challenge device which restricts the flow of sterilant to the indicator using a long and/or tortuous path. While such process challenge devices have been useful, they are not always been convenient to use and/or they have not always provided a close correlation with inactivation of every microorganism present on and/or inside the product or article exposed to the sterilization process.

Accordingly, there continues to be an interest in and a need for challenge devices that are convenient to use and that provide a better correlation between the indication of complete sterilization and the actual complete sterilization of a product or article exposed to the same sterilization process.

### SUMMARY

A process challenge device comprising a self-contained sterilization process biological indicator is now provided, therein with everything needed to rapidly assess the effectiveness of a sterilization process by enabling the detection of germination and/or outgrowth of viable test microorganisms or the detection of enzyme activity, if present, in the self-contained sterilization process biological indicator after exposing the biological indicator to a sterilization process. Advantageously, this discovery provides its user with a process challenge device comprising the self-contained biological indicator wherein the indicator does not have to be removed from the process challenge device before it is activated and observed for detection of viable test microorganisms or active enzyme.

In one aspect, the present disclosure provides a process challenge device for verifying efficacy of a sterilization process. The device can comprise a casing formed of a sterilant vapor-impervious material, the casing forming a cavity with at least one opening that places the cavity in vapor communication with an ambient environment in which the device is disposed; and a self-contained biological indicator operatively connected to the casing. The biological indicator comprises a housing comprising at least one wall, the housing having an open end and an interior space; a plurality of viable test microorganisms or an active enzyme, the test microorganisms or the active enzyme being disposed in the interior space of the housing; a sterilant pathway extending from ambient through the housing to the test microorganisms or the active enzyme; a detection reagent for detecting the viable test microorganisms or the active enzyme, the detection reagent being disposed in the interior space; and an aqueous liquid disposed in an openable container, the aqueous liquid being in selective communication with the viable test microorganisms or active enzyme. The detection reagent can be convertible by the test microorganisms or the active enzyme from a first state to a second state that is distinguishable from the first state. A first portion of the self-contained biological indicator is disposed in the cavity of the casing and a second portion of the self-contained biological indicator is disposed outside the casing. The first portion of the biological indicator comprises the open end. The second portion of the biological indicator comprises a part of the at least one wall through which the aqueous liquid and detection reagent can be observed.

In any embodiment, the process challenge device further comprises an activation support member disposed in the cavity of the casing, wherein the activation support member is positioned proximate the open end of the biological indicator. In some embodiments, the activation support member has a first end moveably disposed in the cavity of the casing, wherein the activation support member has a second end disposed outside the casing, wherein the first end can be moved into contact with the biological indicator. In any of the above embodiments, the casing can comprise an elongated lumen structure, wherein the at least one opening is disposed in the lumen structure. In any of the above embodiments, the biological indicator further can comprise a cap, wherein the cap is slidably attached to the housing so that, when the cap moved from a first position to a second position, it closes the open end of the housing.

In another aspect, the present disclosure provides a method of assessing efficacy of a sterilization process. The method can comprise providing a sterilant gas to an opening of a casing of a process challenge device; wherein the casing is operatively connected to a self-contained biological indicator; wherein the sterilant gas must pass through at least a portion of the casing to contact the self-contained biological indicator; wherein the biological indicator comprises a housing that forms an interior space, a plurality of viable test microorganisms or an active enzyme disposed in the interior space, a detection reagent for detecting the viable test microorganisms or the active enzyme wherein the detection reagent is disposed in the interior space, a sterilant pathway extending from ambient through the housing to the interior space, and an aqueous liquid disposed in the interior space, the aqueous liquid being in selective communication with the viable test microorganisms or active enzyme. The process challenge device is configured so that the biological indicator can be activated and analyzed without detaching the biological indicator from the casing. The efficacy of the sterilization process is determined by a presence of a threshold quantity of the second state in the biological indicator after the biological indicator is activated.

In any implementation of the method, activating the biological indicator can comprise applying a mechanical force against the biological indicator to move a first part of the biological indicator relative to a second part of the biological indicator. In some implementations of the method, the process challenge device further can comprise a plunger, wherein activating the biological indicator can comprise applying a mechanical force to the plunger to move a first part of the biological indicator relative to a second part of the biological indicator.

Providing a sterilant gas comprises providing the sterilant gas at a predefined temperature for a predefined period of time. The method further can comprise, without removing the biological indicator from the process challenge device or without detaching the biological indicator from the process challenge device, activating the biological indicator by placing the viable test microorganism or active enzyme in contact with the reagent; and without removing the biological indicator from the process challenge device or without detaching the biological indicator from the process challenge device, analyzing the biological indicator to detect a change in the detection reagent from a first state to a second state; wherein the efficacy is determined by a presence of a threshold quantity of the second state in the biological indicator after the biological indicator is activated.

In any implementation of the method, activating the biological indicator can comprise applying a mechanical force to the biological indicator to move a first part of the biological indicator relative to a second part of the biological indicator. In certain implementations of the method, the process challenge device further comprises a plunger, wherein applying a force comprises moving the plunger to apply the force. In any of the above implementations of the method, moving a first part of the biological indicator relative to a second part of the biological indicator comprises opening the openable container comprises opening the frangible container. In any of the above implementations, the method further comprises before providing the sterilant gas, positioning the process challenge device in a sterilization chamber of an automated sterilizer; wherein providing the sterilant gas to the opening comprises operating the automated sterilizer to provide the sterilant gas into the sterilization chamber. In any of the above implementations of the method, analyzing the biological indicator can comprise visually observing the biological indicator to detect the change. In any of the above implementations of the method, analyzing the biological indicator can comprise placing a portion of the biological indicator into an optical device that detects the second state of the detection reagent.

Additional details of these and other embodiments are set forth in the accompanying drawings and the description below. Other features, objects and advantages will become apparent from the description and drawings, and from the accompanying claims.

Herein, the terms “biological sterilization process indicators”, “sterilization process biological indicator”, “sterilization process indicator”, “biological indicator”, “BI”, “indicator”, “self-contained biological indicator”, and “SCBI” are used interchangeably.

The numbers, E5, E6, and E7 are used interchangeably herein with 10⁵, 10⁶, and 10⁷, respectively.

The term “comprising” and variations thereof (e.g., comprises, includes, etc.) do not have a limiting meaning where these terms appear in the description and claims.

As used herein, “a”, “an”, “the,” “at least one,” and “one or more” are used interchangeably, unless the context clearly dictates otherwise.

Also, herein, the recitations of numerical ranges by endpoints include all numbers subsumed within that range (e.g., 500 to 7000 nm includes 500, 530, 551, 575, 583, 592, 600, 620, 650, 700, etc.).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “actuatable container”, as used herein, refers to a container that can be actuated, when desired, to release contents therein. The container can be actuated, for example, the container can by dislodging or removing a plug, by actuating a valve to change it from a “closed” state to an “open” state, or by otherwise breaching at least a portion of the container.

The term “frangible container” refers to any container that can be acted upon to release its contents, for example by breaking it, puncturing it, shattering it, cutting it, etc.

All scientific and technical terms used herein have meanings commonly used in the art unless otherwise specified. The definitions provided herein are to facilitate understanding of certain terms used frequently in this application and are not meant to exclude a reasonable interpretation of those terms in the context of the present disclosure.

The term “adjacent” refers to the relative position of two elements, such as, for example, two layers, that are close to each other and may or may not be necessarily in contact with each other or that may have one or more layers separating the two elements as understood by the context in which “adjacent” appears.

“Sterilization process”, as used herein, refers to a process that intentionally contacts an article with a vapor sterilant under preselected conditions (e.g., concentration of sterilant, temperature, humidity, contact time) to render the surface(s) of the article free of viable microorganisms. Such processes may be conducted in a sterilization chamber of an automated sterilizer, wherein the automated sterilizer controls at least one of the preselected conditions.

A “self-contained biological indicator”, as used herein, refers to a biological sterilization indicator that comprises all of the components (e.g., a source of biological activity such as, for example, test microorganisms, spores, or an active enzyme; a detection reagent for detecting a viable spore or microorganism or for detecting the active enzyme; a suitable solvent in which to dissolve or suspend the source of biological activity and the reagent (e.g., water or an aqueous buffer) so that the source can react with the reagent; and, optionally, a nutrient to facilitate germination and/or growth of a test microorganism or spore (if present) needed to assess the survival of the test microorganism or the active enzyme after the biological indicator is exposed to a vapor sterilant in a sterilization process. Test microorganisms used in a self-contained biological indicator can include, but are not limited to, at least one of Geobacillus stearothermophilus, Bacillus stearothermophilus, Bacillus subtilis, Bacillus atrophaeus, Bacillus megaterium, Bacillus coagulans, Clostridium sporogenes, Bacillus pumilus, or combinations thereof.

“Vapor sterilant” or “sterilant gas”, as used herein, are used interchangeably and refer to chemical agents that are dispersed in vapor form (e.g., as a gas and/or plasma) in a sterilizer during a process intended to inactivate microorganisms e.g., bacteria, spores, viruses, prions). Non-limiting examples of vapor sterilants include ethylene oxide, hydrogen peroxide, steam, ozone, and combinations thereof. Unless otherwise indicated, all numbers in the description and the claims expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

The above summary of the present invention is not intended to describe each disclosed embodiment or every implementation of the present invention. The description that follows more particularly exemplifies illustrative embodiments.

### BRIEF DESCRIPTION OF DRAWINGS

FIG. 1A is a cross-sectional side view of one embodiment of a self-contained biological indicator that can be used to produce a process challenge device according to the present disclosure.

FIG. 1 B is an exploded perspective view of the self-contained biological indicator of FIG. 1A.

FIG. 2A is a plan view of one embodiment of a process challenge device according to the present disclosure.

FIG. 2B is a cross-sectional side view of the process challenge device of FIG. 2A.

FIG. 2C is a view of one end of the process challenge device of FIG. 2A.

FIG. 2D is a view of another end of the process challenge device of FIG. 2A.

FIG. 3 is a perspective view of one embodiment of a self-contained biological indicator;

FIG. 4A is a plan view, partially in section, of an alternative embodiment of a process challenge device according to the present disclosure.

FIG. 4B is a cross-sectional side view of the process challenge device of FIG. 4A.

FIG. 4C is a view of one end of the process challenge device of FIG. 4A.

FIG. 5A is a plan view, partially in section, of an alternative embodiment of a process challenge device according to the present disclosure wherein the chamber of the process challenge device comprises an elongated lumen.

FIG. 5B is a view of one end of the process challenge device of FIG. 5A.

FIG. 6 is a photograph of the process challenge device comprising a chemical integrator device as described in Example 1.

FIG. 7 is a photograph of the process challenge device of FIG. 6 before the chemical integrator device was inserted therein.

FIG. 8 shows the process challenge device of FIG. 6 being analyzed by an automated reader.

FIG. 9A is a photograph of one embodiment of a process challenge device comprising at least one elongated lumen.

FIG. 9B is a photograph of an alternative embodiment of a process challenge device comprising at least one elongated lumen.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “supported,” and “coupled” and variations thereof are used broadly and encompass both direct and indirect supports and couplings. Further, “connected” and “coupled” are not restricted to physical or mechanical connections or couplings. It is to be understood that other embodiments may be utilized, and structural or logical changes may be made without departing from the scope of the present disclosure. Furthermore, terms such as “front,” “rear,” “top,” “bottom,” and the like are only used to describe elements as they relate to one another but are in no way meant to recite specific orientations of the apparatus, to indicate or imply necessary or required orientations of the apparatus, or to specify how the invention described herein will be used, mounted, displayed, or positioned in use.

Medical device reprocessor define a process challenge device (hereinafter, “PCD”) is a “test device intended to provide a challenge to the sterilization process that is equal to or greater than the challenge posed by the most difficult item routinely processed.” (MDRAO; Medical Device Reprocessing Manual, 4^{th} ed.; 2017 Brown Book Company; Scarborough, Ontario). In practice, a PCD is used to confirm that a sterilizer has effectively sterilized ALL items processed in that cycle. One example of a PCD is a package containing a Biological Indicator (“BI”) comprising spores and a Class V indicator strip (e.g., chemical integrator device, or “CI”) inserted inside the package. This package is then placed in a routine sterilizer load with other instrument pouches or in a wrapped cassette to challenge and test the sterilization process. If the BI shows that all the spores have been killed and the CI indicates all critical variables have met or exceeded the performance requirements and the sterilizer indicates that all physical parameters (time, temperature, and pressure) have been verified, you can be confident that the sterilization process has been effective and that the sterilizer is working properly. The three types of PCDs most commonly encountered in practice are the following: 1. Air detection PCD (Bowie-Dick test pack), 2. A biological PCD test pack, and 3. A chemical indicator PCD test pack.

The present disclosure generally relates to a sterilization indicator, and particularly, to a process challenge device comprising a chemical indicator and/or a biological sterilization indicator. Embodiments of the biological sterilization indicator of the present disclosure are self-contained and can be used to determine the lethality of a sterilizing process. The present disclosure generally relates to the construction and use of the process challenge device comprising a biological sterilization indicator that can be actuated and analyzed without opening the process challenge device and without disconnecting or removing the biological sterilization from the process challenge device. Advantageously, some embodiments of the process challenge device of the present disclosure may be constructed of materials that permit re-use of the casing (with a new biological indicator operatively attached thereto) in one or more subsequent sterilization processes.

Process challenge devices of the present disclosure are used to assess the efficacy of sterilization processes that use vapor sterilants to inactivate viable microorganisms present on and/or inside the articles to be sterilized. Such process challenges devices include a biological sterilization indicator and a casing as described herein.

Process challenge devices of the present disclosure comprise a self-contained biological indicator. Self-contained biological indicators are well known in the art of sterilization processes and are available commercially from a number of companies including, for example, 3M Company, Steris Corporation, and ASP. Most commercially-available self-contained biological indicators share a number of common structural features that make them suitable for use in a process challenge device of the present disclosure.

A variety of self-contained biological indicators suitable for use in a process challenge device or method of the present disclosure are known in the art. The self-contained biological indicator can be used to determine the lethality of a sterilizing process that uses a vapor sterilant. Non-limiting examples of suitable self-contained biological indicators are described in U.S. Pat. Nos. 6,623,955; 9,322,046; and 6,623,955; which are incorporated herein by reference in their entirety.

In certain embodiments, the self-contained biological indicator can comprise an active enzyme. “Active enzyme”, as used herein, refers to an enzyme that catalyzes a reaction with a detection reagent (e.g., a fluorogenic or chromogenic enzyme substrate) to form a detectable product as described, for example, in U.S. Pat. No. 9,322,046. In some embodiments, the source of active enzyme can be (1) the purified, isolated enzyme derived from an appropriate microorganism; (2) a microorganism to which the enzyme is indigenous or added by genetic engineering; and/or (3) a microorganism to which the enzyme has been added during sporulation or growth, such that the enzyme is incorporated or associated with the microorganism, e.g., an enzyme added to a spore during sporulation which becomes incorporated within the spore. In some embodiments, the microorganisms which may be utilized as the source of the active enzyme include bacteria or fungi in either the spore or vegetative state. In some embodiments, the enzyme source includes Bacillus, Clostridium, Neurospora, Candida, or a combination of such species of microorganisms.

A process challenge device of the present disclosure can be used for verifying efficacy of a sterilization process. The process challenge device comprises a casing formed of a sterilant vapor-impervious material and a self-contained biological indicator operatively connected to the casing as described herein. The casing forms a cavity with at least one opening that places the cavity of the casing in vapor communication with an ambient environment in which the device is disposed.

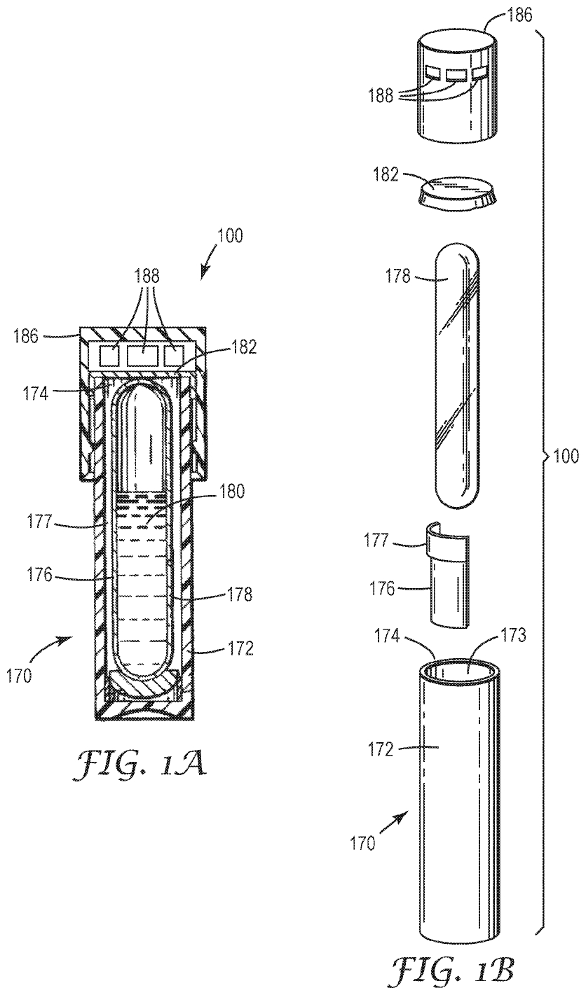

Turning to the drawings, FIG. 1 shows one embodiment of a biological indicators (biological indicator 100) that is suitable as a biological indicator for use in process challenge devices and methods of the present disclosure.

Biological indicator 100 is described in U.S. Pat. No. 6,623,955. The biological indicator 100 comprises a first portion 101 and a second portion 102. The biological indicator 100 further comprises inter alia a housing 170 that forms an interior space 173. The housing 170 is formed of gas nonabsorptive and liquid impermeable walls 172 with an open end 174. The biological indicator further comprises an openable (e.g., frangible) inner container 178 disposed in the interior space 173, the inner container containing an aqueous liquid 180; an optional gas-transmissive, bacteria-impermeable closure member 182, which may be attached to (e.g., adhesively attached to) the open end 174 of the housing 170 and/or the closure member may be held in place by an optional cap 186. The cap 186 comprises one or more vent 188 that provides fluid (e.g., vapor) communication between ambient and the interior space 173 of the housing 170.

The biological indicator further comprises a detection reagent (not shown) for detecting a viable spore or microorganism or for detecting an active enzyme. A “detection reagent”, as used herein, refers to a substance that can react with the source of biological activity (e.g., test microorganism, spore, or active enzyme) to form a detectable product directly or indirectly. Non-limiting examples of suitable detection reagents include nutrients (e.g., a sugar) that can be converted (e.g., by fermentation) to an acidic or basic end-product which presence can be detected indirectly by a pH indicator. Other non-limiting examples of suitable detection reagents include chromogenic enzyme substrates and fluorogenic enzyme substrates that can be acted on by an enzyme activity to form a detectable (e.g., by spectrophotometry or fluorometry) product. The detection reagent may be disposed (e.g., as a powder or a tablet) in the interior space 173. Alternatively, the detection reagent may be dissolved and/or suspended in the aqueous liquid 180. The detection reagent is convertible by the test microorganisms or the active enzyme from a first state (e.g., colorless) to a second state (e.g., colored) that is distinguishable from the first state. Detection reagents include, for example, chromogenic and fluorogenic enzyme substrates. Detection reagents also include metabolizable nutrients (e.g., glucose) coupled with a pH indicator, for example. A number of suitable detection reagents are described in U.S. Pat. Nos. 6,623,955 and 9,322,046; which are incorporated herein by reference in their entirety. Optionally, a nutrient growth medium (not shown) to facilitate germination and/or growth of the spore or test microorganism may be disposed in the interior space 173 of the housing 170 (e.g., as a powder or a tablet) or may be dissolved and/or suspended in the aqueous liquid 180.

The biological indicator 100 further comprises a plurality of viable test microorganisms (not shown) or another source of active enzyme (not shown) disposed in the interior space 173 of the housing 170, optionally, on a carrier 177. Optionally, the carrier is attached to a wick strip 176 by, for example, adhesive or heat sealing. The wick strip 176 can be made from any water-absorbent material, such as filter paper, cloth, or rayon. Additionally, the wick strip can be constructed of a combination of materials such as paper secured to a plastic or glass backing strip. Preferably wick strip 176 is prepared from polyethylene coated paper. The dimensions of the wick strip 176 and the placement of the carrier 177 on the wick strip 76 are such that when the inner-container is ruptured, the aqueous liquid 180 therein is contained within the lower portion of the housing 170 and below the carrier 177. The aqueous solution 180 travels up the wick strip 176 to test microorganisms or active enzyme disposed on the carrier 177. The growing microorganisms and/or enzyme-modified product concentrates on the carrier 177 and its presence is detected in a shorter period of time than would be the case if the carrier were exposed to the entire solution present in the inner container 178. A fluid pathway (not shown) extends through the interior space 173 of the housing 170 from the open end 174 of the housing 170 to the carrier 177 holding the test microorganisms or the active enzyme. The fluid pathway provides access for a vapor sterilant to permeate the interior of the housing and contact the test microorganisms or the active enzyme, thereby potentially causing loss of viability and/or inactivation of active enzyme. The open end 174 of the housing 170 is disposed in the first portion 101 of the biological indicator 100.

In some embodiments described hereinbelow, the cap may be part of a system that is used to open the inner container when the biological indicator is activated (e.g., after the process challenge device has been exposed to a sterilization process.

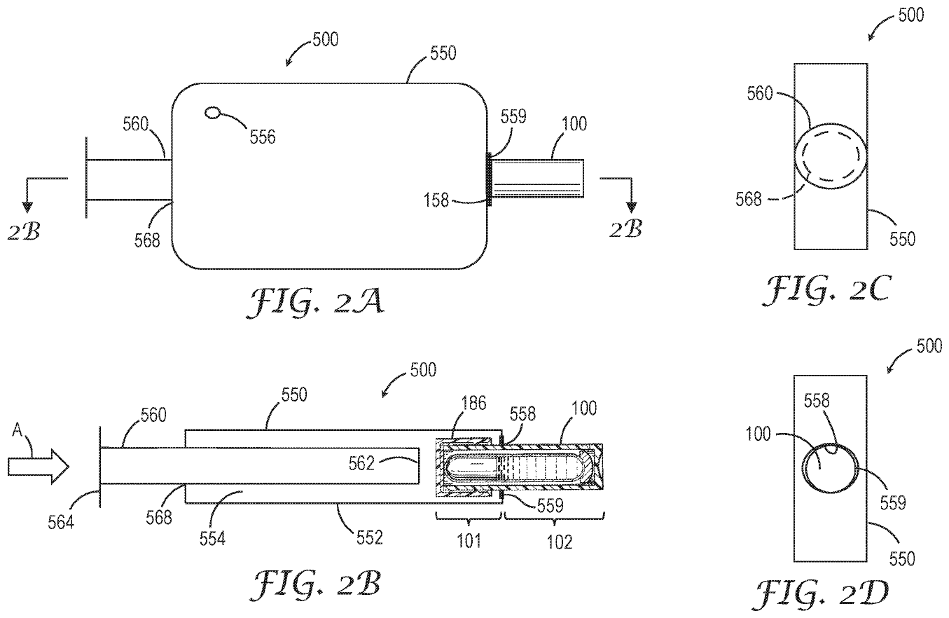

FIGS. 2A-D show various views of one embodiment of a process challenge device 500 according to the present disclosure. The device 500 comprises a self-contained biological indicator 100 operatively connected to a casing 550. The casing 550 comprises at least one casing wall 552 that is formed of a material that is impervious to the vapor sterilant that is used in the sterilization process that the process challenge device is used to monitor.

The casing can be constructed from a variety of materials that do not substantially disintegrate when exposed to the conditions of a sterilization process. The materials include, for example, polymeric materials such as polypropylene, polyethylene, polyethylene terephthalate (PET), polycarbonate, polyolefin, polystyrene, polyacrylamide, polymethacrylate, poly(methyl)methacrylate, polyimide, polyester, polyethylene, terephthalate, polybutylene terephthalate, polyvinylchloride, or a copolymer or a mixture thereof. In some embodiments, the casing wall 552 may include glass, ceramic, metal, or a combination thereof. For example, the casing wall 552 may be prepared from a polymeric material combined with another material, such as a polymeric film co-extruded or laminated onto a metallic film. The casing 550 forms a cavity 554 with at least one opening 556 that places the cavity 554 in vapor communication with an ambient environment in which the device 500 is disposed. The casing further comprises a first aperture 558 in which the biological indicator 100 is disposed so that the first portion (shown in FIG. 2B) of the biological indicator 100, which includes the open end (174) of the biological indicator, is disposed in the cavity 554 of the casing 550 and the second portion of the biological indicator 100 is disposed outside the casing 550.

The casing 550 can be fabricated from a variety of vapor-sterilant-impervious materials (e.g., a thermoplastic polymer) known in the art of PCDs and by using processes (e.g., thermal molding) that are well-known in the art. In some embodiments, the casing 550 comprises two parts that are sealingly joined together by an adhesive or by thermal welding, for example.

The biological indicator 100 can be operatively connected to the casing 550 in a variety of ways. “Operatively connected”, as used herein, means that the biological indicator 100 is attached to the casing 550 in a way that: 1) permits the biological indicator 100 to be activated without detaching or removing the biological indicator from the casing, 2) permits observation of the biological indicator 200 after activation to detect whether at least one of the test microorganisms is viable and/or at least some of the active enzyme is active, and 3) does not provide fluid communication via the first aperture between the cavity of the casing and the ambient environment outside the casing.

In the illustrative embodiment of FIGS. 2A-D, the biological indicator 100 is operatively connected to the casing 550 by inserting the first portion 101 of the biological indicator into the casing through the first aperture 558 into the cavity to form a tight fit between the first aperture and the biological indicator. In the illustrated embodiment, a gasket 559 disposed in the first aperture 558 between the casing 550 and the biological indicator 100 provides a tight seal to hold the biological indicator in place and to prevent fluid (e.g., vapor) ingress or egress through the first aperture 558. The second portion 102 of the biological indicator 100 protrudes from the casing, making it possible to actuate the biological indicator using techniques that are well known in the art of self-contained biological indicators (e.g., by applying force against wall of the indicator (and the inner container disposed therein) until the inner container fractures and releases the aqueous liquid).

During the normal use of some biological indicators, including the biological indicator 100 of the illustrated embodiment of FIGS. 2A-D, the cap is moved from an “open” position (which provides a vapor pathway into the biological indicator) to a “closed” position which prevents vapor or liquid from entering or exiting the biological indicator) prior to activating the biological indicator. Accordingly, a process challenge device of the present disclosure optionally comprises an activation support member 560 that can be used to assist operator while activating the biological indicator (e.g., after the process challenge device has been used to monitor a sterilization process). The activation support member 560 of the illustrated embodiment of FIGS. 2A-D has a first end 562 and a second end 564 opposite the first end. Mechanical force (e.g., manual pressure) can be exerted against the second end 564, causing the activation support member 560 to move in the direction of arrow “A” until the first end 562 contacts the cap 186 of the biological indicator 100 and moves a first part of the biological indicator (e.g., the cap 186) relative to a second part of the biological indicator (e.g., the housing 170), which thereby places the biological indicator in the “closed” position. After the cap 186 is moved into the closed position, the biological indicator 100 can be activated as disclosed herein. In any embodiment, the activation support member 560 can take the form of a plunger that is partially inserted into the casing 550 through a second aperture 568. The activation support member 560 and the second aperture 568 are dimensioned to provide a tight fit while permitting movement of the activation support member 560 when a mechanical force is applied to the second end 564. The activation support member 560 can be used to help close the cap 184 of the biological indicator 100 and/or it can be used to support the biological indicator (e.g., hold it in place relative to the casing) while the biological indicator is activated.

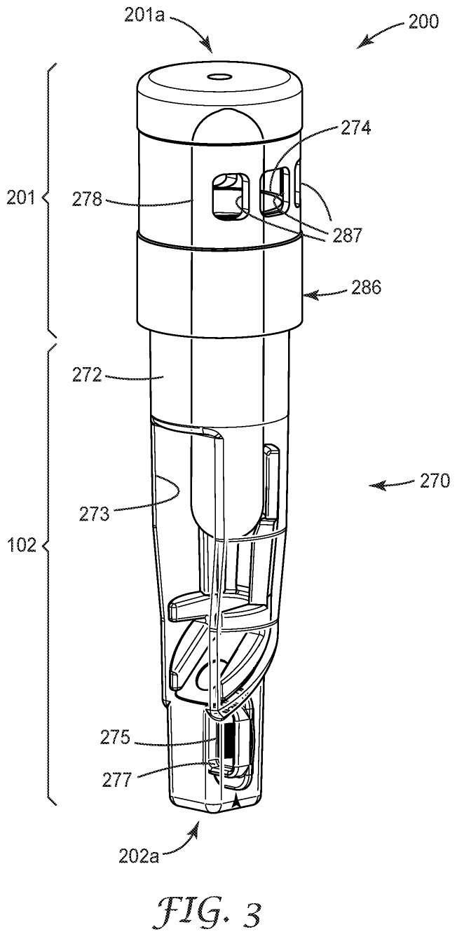

FIG. 3 shows another embodiment of a self-contained biological indicator 200 that is suitable for use in the process challenge devices and methods of the present disclosure. Biological indicator 200 is described in U.S. Pat. No. 9,322,046, which is incorporated herein by reference in its entirety.

The biological indicator 200 comprises inter alia a first portion 201 with a first end 201a and a second portion 202 with a second end 202a. The biological indicator further comprises a housing 270 that comprises at least one gas nonabsorptive and liquid impermeable wall 272 formed of sterilant vapor-impervious material. The housing 270 has an interior 273 in which other components of the indicator are disposed. The biological indicator 200 also comprises a carrier 277 disposed in the interior of the housing 270. Test microorganisms (e.g., spores 275) or an active enzyme (not shown) are disposed on the carrier 277. A fluid pathway (not shown) extends through the interior of the housing 270 from the open end 274 of the housing 270 to the carrier 277 holding the test microorganisms or the active enzyme. The fluid pathway provides access for a vapor sterilant to permeate the interior of the housing and contact the test microorganisms or the active enzyme, thereby potentially causing loss of viability and/or inactivation of active enzyme. The open end 274 of the housing is disposed in the first portion 201 of the biological indicator 200.

Housing 270 also holds therein an aqueous liquid disposed in an openable (e.g., frangible) inner container 278. Thus, the aqueous liquid (not shown) in the inner container 278 is in selective communication with the test microorganisms or the active enzyme in that when the openable inner container 278 is closed and intact, the aqueous liquid is isolated from the test microorganisms or the active enzyme and when the openable inner container 278 is opened (e.g., by fracturing it under pressure), the aqueous liquid can contact the test microorganisms and the active enzyme.

Optionally, the biological indicator 200 includes a cap 286 that covers the open end 274 of the housing 270. The cap 286 comprises one or more vent (287) that provides fluid (e.g., vapor) communication between ambient and the interior of the housing 270. In some embodiments described hereinbelow, the cap may be part of a system that is used to open the inner container 278 when the biological indicator is activated (e.g., after the process challenge device has been exposed to a sterilization process. The biological indicator 200 can also comprise an optional gas-transmissive, bacteria-impermeable closure member (not shown in FIG. 3) e.g., occluding vents 287 to prevent passage of bacterial contaminants into the interior of the housing 270 during use and/or storage.

A biological indicator suitable for a process challenge device or method of the present disclosure further includes a detection reagent as described hereinabove for detecting the viable test microorganisms or the active enzyme. In the illustrated embodiment of FIG. 3, the detection reagent is dissolved or suspended in the aqueous liquid (not shown) contained in the openable inner container 278. It is contemplated that, in some embodiments (not shown), the detection reagent may be disposed in the interior of the housing (e.g., as a dry powder or a dissolvable capsule) and not disposed in the openable inner container. In addition, the biological indicator 200 may include a nutrient as described hereinabove. The biological indicator 200 further comprises a number of structures that act cooperatively to facilitate opening the inner container 278 by urging the cap 286 toward the second portion 202 of the housing 270 as described in U.S. Pat. No. 9,322,046.

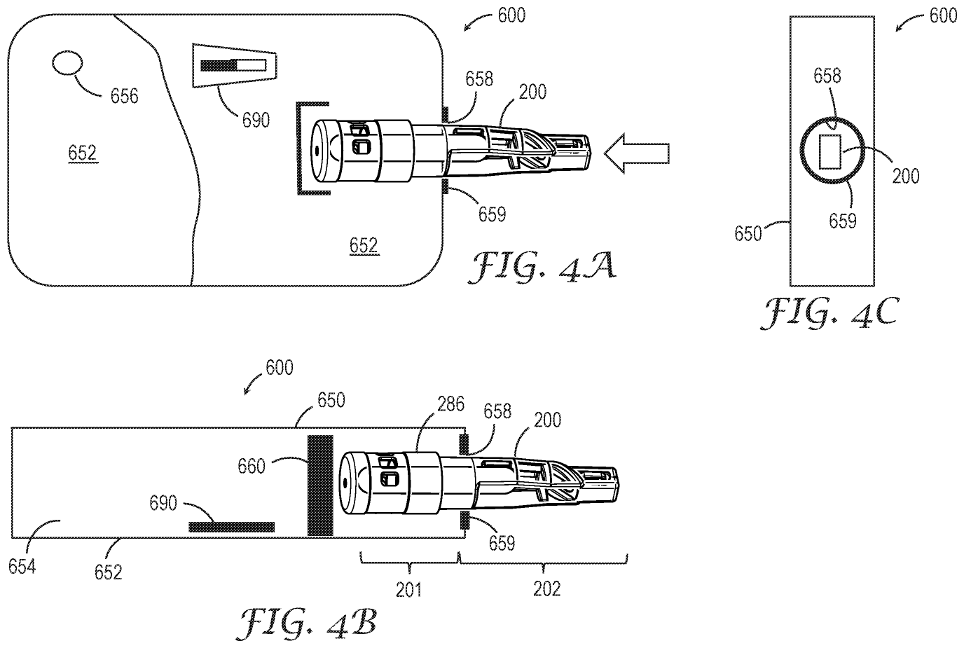

FIGS. 4A-C show various views of one embodiment of a process challenge device 600 according to the present disclosure. The device 600 comprises a self-contained biological indicator (e.g., biological indicator 200 as described above) operatively connected to a casing 650. The casing 650 comprises at least one casing wall 652 that is formed of a material that is impervious to the vapor sterilant that is used in the sterilization process that the process challenge device is used to monitor. The casing 650 forms a cavity 654 with at least one opening 656 that places the cavity 654 in vapor communication with an ambient environment in which the device 600 is disposed. The casing 650 further comprises a first aperture 658 in which the biological indicator 200 is disposed so that the first portion 201 (shown in FIG. 2B) of the biological indicator 200, which includes the open end 274 of the biological indicator, is disposed in the cavity 654 of the casing 650 and the second portion 202 of the biological indicator 200 is disposed outside the casing 650.

The casing 650 can be fabricated from a variety of vapor-sterilant-impervious materials (e.g., a thermoplastic polymer) known in the art of PCDs and by using processes (e.g., thermal molding) that are well-known in the art. In some embodiments, the casing 650 comprises two parts (e.g., walls) that are sealingly joined together by an adhesive or by thermal welding, for example.

The biological indicator 200 can be operatively connected to the casing 650 in a variety of ways. “Operatively connected”, as used herein, means that the biological indicator 200 is attached to the casing 650 in a way that: 1) permits the biological indicator 200 to be activated without detaching or removing the biological indicator from the casing, 2) permits observation of the biological indicator 200 after activation to detect whether at least one of the test microorganisms is viable and/or at least some of the active enzyme is active, and 3) does not provide fluid communication via the first aperture 658 between the cavity of the casing and the ambient environment outside the casing.

In the illustrative embodiment of FIGS. 4A-C, the biological indicator 200 is operatively connected to the casing 650 by inserting the first portion 201 of the biological indicator into the casing through the first aperture 658 into the cavity to form a tight fit between the casing and the biological indicator. In the illustrated embodiment, a gasket 659 disposed in the first aperture 658 between the casing 650 and the biological indicator 200 provides a tight seal to hold the biological indicator in place and to prevent fluid (e.g., vapor) ingress or egress through the first aperture. The second portion 202 of the biological indicator 200 protrudes from the casing 650, making it possible to actuate the biological indicator using techniques that are well known in the art of self-contained biological indicators (e.g., by applying force against wall of the indicator (and the inner container disposed therein) until the inner container fractures and releases the aqueous liquid).

During the normal use of some biological indicators, including the biological indicator 200 of the illustrated embodiment of FIGS. 4A-C, the cap is moved from an “open” position (which provides a vapor pathway into the biological indicator) to a “closed” position which prevents vapor or liquid from entering or exiting the biological indicator) prior to activating the biological indicator. Accordingly, the process challenge device of the illustrated embodiment of FIGS. 4A-D comprises an activation support member 660 that can be used to assist operator while activating the biological indicator (e.g., after the process challenge device has been used to monitor a sterilization process). The activation support member 660 of the illustrated embodiment of FIGS. 2A-D has a first end 562 and a second end 564 opposite the first end. Mechanical force (e.g., manual pressure) can be exerted against the second end 564, causing the activation support member 560 to move in the direction of arrow “A” until the first end 562 contacts the cap 186 of the biological indicator 100 and moves a first part of the biological indicator (e.g., the cap 186) relative to a second part of the biological indicator (e.g., the housing 170), which thereby places the biological indicator in the “closed” position. The activation support member 660 of the illustrated process challenge device 600 is a structure that can either be molded one of the walls of the casing 650, attached to the interior of the casing, or supported by a portion of the casing such that the activation support member remains substantially in place when a mechanical force (e.g., manual pressure) is exerted upon the second portion 202 of the biological indicator 200 in the direction of arrow “B”. The force causes the second portion 202 of the biological indicator 200 to slide into the casing 650, thereby urging the cap 286 against the housing of the biological indicator thereby sealing the biological indicator closed. After the cap 286 is moved into the closed position, the biological indicator 200 can be activated as disclosed herein. The activation support member 660 can be used to help close the cap 284 of the biological indicator 200 and/or it can be used to support the biological indicator (e.g., hold it in place relative to the casing) while the biological indicator is activated. In some embodiments (not shown), the activation support member comprises a part of the at least one wall of the casing.

It is contemplated that a process challenge device comprising a movable activation support member such as, for example, the illustrated embodiment of FIGS. 2A-D may comprise a biological indicator similar to the illustrated embodiment of FIG. 3. In these embodiments (not shown), the movable activation support member may facilitate two operations: 1) closing/sealing the biological indicator and 2) actuating the biological indicator by causing the inner compartment to rupture.

In any embodiment, a process challenge device of the present disclosure further can comprise a chemical integrator device (e.g., a 3M™ COMPLY™ STERIGAGE™ Steam Chemical Integrator) disposed in the cavity of the process challenge device. Whereas the self-contained biological indicator of the present process challenge device can indicate whether the sterilization process is lethal to test microorganisms and/or active enzymes, the chemical integrator device can indicate whether certain parameters (e.g., temperature, time of exposure to temperature) associated with efficacious sterilization processes have been achieved in the process challenge device. FIGS. 4A-B show one embodiment of a process challenge device 600 comprising a chemical integrator device 690 disposed in the cavity 654 of the casing 650 according to the present disclosure. In these embodiments, the at least one wall 652 of the casing 650 has sufficient optically transmissibility (e.g., clarity) to permit observation and analysis of the chemical integrator device 690 without having to remove it from the casing 650.

In another aspect, the cavity of the casing of a process challenge device of the present disclosure may optionally comprise an elongated lumen that comprises the opening into the cavity. The elongated lumen can mimic lumened medical devices (e.g., endoscopes) and, thus, provide a more realistic challenge to a sterilization process than a biological indicator that does not comprise an elongated lumen. In addition, the length and inner diameter of the lumen used to construct a process challenge device according to the present disclosure each can be varied in order to provide a more-resistant or less-resistant challenge as desired by the operator.

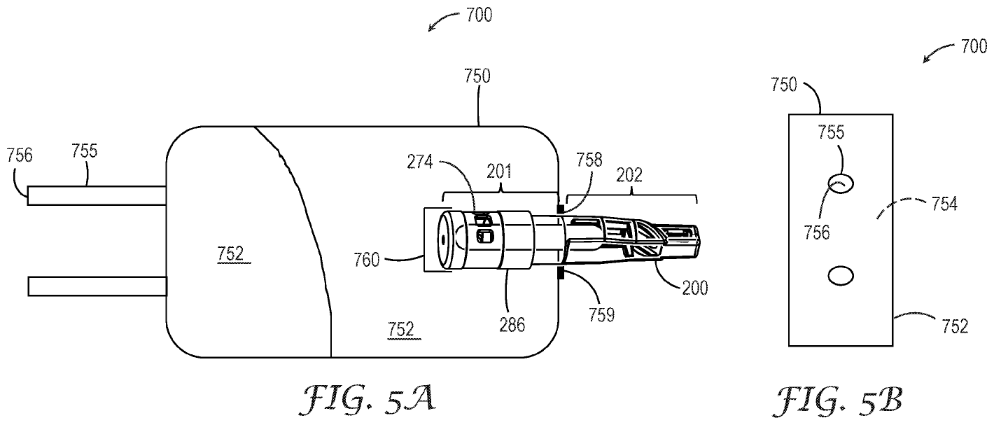

FIGS. 5A-B show one embodiment of a process challenge device 700 comprising a casing 750 that includes at least one elongated lumen according to the present disclosure. The device 700 comprises a casing comprises at least one casing wall 752 that is formed of a material that is impervious to the vapor sterilant that is used in the sterilization process that the process challenge device is used to monitor, as described above. The casing 750 forms a cavity 754 comprising an elongated lumen 755 having an opening 756 that places the cavity 754 in vapor communication with an ambient environment in which the device 600 is disposed. The casing 750 further comprises a first aperture 758 in which a biological indicator is disposed. The biological indicator can be any suitable biological indicator (e.g., biological indicator 200) as described herein. The biological indicator 200 is disposed so that a first portion 201 (shown in FIG. 2B) of the biological indicator 200, which includes an open end 274 of the biological indicator, is disposed in the cavity 754 of the casing 750 and a second portion 202 of the biological indicator 200 is disposed outside the casing 750.

In the illustrative embodiment of FIGS. 5A-B, the biological indicator 200 is operatively connected to the casing 750 by inserting the first portion 101 of the biological indicator into the casing through the first aperture 758 into the cavity to form a tight fit between the first aperture and the biological indicator. In the illustrated embodiment, a gasket 759 disposed in the first aperture 758 between the casing 750 and the biological indicator 200 provides a tight seal to hold the biological indicator in place and to prevent fluid (e.g., vapor) ingress or egress through the first aperture 758. The second portion 202 of the biological indicator 200 protrudes from the casing, making it possible to actuate the biological indicator using techniques that are well known in the art of self-contained biological indicators (e.g., by applying force against wall of the indicator (and the inner container disposed therein) until the inner container fractures and releases the aqueous liquid).

During the normal use of some biological indicators, including the biological indicator 200 of the illustrated embodiment of FIGS. 5A-B, the cap is moved from an “open” position (which provides a vapor pathway into the biological indicator) to a “closed” position which prevents vapor or liquid from entering or exiting the biological indicator) prior to activating the biological indicator. Accordingly, a process challenge device of the present disclosure optionally comprises an activation support member 760 as described hereinabove. The activation support member 760 of the illustrated process challenge device 700 is a structure that can either be molded one of the walls of the casing 750, attached to the interior of the casing, or supported by a portion of the casing such that the activation support member remains substantially in place when mechanical force (e.g., manual pressure) is exerted upon the second portion 202 of the biological indicator 200 as described hereinabove. The force causes a first part (e.g., the housing 270 of the biological indicator 200) to slide into the casing 750, and thereby move relative to a second part (e.g., the cap 286) of the biological indicator and seal the biological indicator closed. After the cap 286 is moved into the closed position, the biological indicator 200 can be activated as disclosed herein.

In any embodiment, a process challenge device of the present disclosure that comprises a cavity that includes an elongated lumen further can comprise a chemical integrator device (not shown in FIGS. 5A-B) as described herein.

In yet another aspect, the present disclosure provides a method of assessing efficacy of a sterilization process. The method comprises i) providing a sterilant gas to an opening of a casing of a process challenge device wherein the casing forms a cavity that is operatively connected to, a self-contained biological indicator. In any embodiment, providing the sterilant gas to the opening comprises providing the sterilant gas (e.g., steam) at a predefined temperature (e.g., 121° C., 132° C., or 135° C. for steam sterilization) for a predefined period of time. In any implementation of the method, the method further comprises a step iv) before providing the sterilant gas, positioning the process challenge device in a sterilization chamber of an automated sterilizer; wherein providing the sterilant gas to the opening comprises operating the automated sterilizer to provide the sterilant gas into the sterilization chamber of the automated sterilizer.

The biological indicator of the process challenge device of the method comprises a housing with an interior; a plurality of viable test microorganisms or an active enzyme disposed in the interior; a detection reagent for detecting the viable test microorganisms or the active enzyme, the detection reagent being disposed in the interior; a fluid pathway extending through the housing to the interior; and an aqueous liquid, the aqueous liquid being in selective communication with the viable test microorganisms or active enzyme. The sterilant gas must pass through at least a portion of the casing to contact the test microorganisms, if present, or the active enzyme, if present. The process challenge device is configured so that the biological indicator can be activated and analyzed without disconnecting the biological indicator from the casing. For example, a portion of the biological indicator can protrude from the casing, making it possible to activate the biological indicator without disconnecting it from the casing, as described herein.

The method further comprises ii) without detaching the biological indicator from the casing, activating the biological indicator by placing the viable test microorganism or active enzyme in aqueous contact with the detection reagent. This step is accomplished by opening (e.g., by fracturing as discussed herein) the container (e.g., the openable inner container described herein) containing the aqueous liquid and thereby releasing the aqueous liquid into the interior space of the biological indicator where the aqueous liquid can facilitate interaction between the test microorganisms (or active enzyme), the detection reagent, and optionally the nutrient, if present, in the biological indicator. In any embodiment of the method, activating the biological indicator can comprise applying a force to the biological indicator to compress a part of the biological indicator.

The method further comprises iii) without detaching the biological indicator from the casing, analyzing the biological indicator to detect a change in the detection reagent from a first state to a second state. In some embodiments, analyzing the biological indicator to detect a change in the detection reagent from a first state to a second state comprises observing the aqueous liquid (e.g., observing visually, spectrophotometrically, fluorometrically) to detect a colored product (e.g., a chromogenic enzyme substrate, a pH indicator) or a fluorescent product. Because a portion of the biological indicator protrudes outside the casing, the liquid contents of the biological indicator can be observed visually or mechanically (e.g., by placing the portion of the biological indicator into an instrument (e.g., an auto-reader)). The efficacy of the sterilization process can be determined by a presence of a threshold quantity of the second state in the biological indicator after the biological indicator is activated. The threshold quantity may simply be a detectable amount (e.g., visually observable or a measurable quantity), or an arbitrary predetermined quantity (e.g., a preset value programmed into an auto-reader, or the threshold quantity may be a measurable increase in the quantity second state as the second state is measured (e.g., by an auto-reader) over a period of time.

Existing methods for using process challenge devices comprising biological indicators to monitor the efficacy of a sterilization process require detaching and/or removing the biological indicator from the device so that the biological indicator can be activated, incubated, and/or analyzed. Advantageously, a method of the present disclosure does not require detachment or removal of the biological indicator from the casing prior to activating, incubating, or analyzing the biological indicator. Because the second portion of the biological indicator extends outside the casing, the biological indicator can be sealed (e.g., by moving the cap relative to the open end of the biological indicator as disclosed herein) and the biological indicator can be activated by opening the inner container as disclosed herein.

In any implementation of the method, analyzing the biological indicator (after activation) can comprise visually observing the second portion of the biological indicator that extends outside the casing of the process challenge device. If any of the test microorganisms or active enzyme survives the sterilization process, the detection reagent will be converted (by the test microorganisms or active enzyme) from the first state to the second state. In certain implementations, this results in, for example, a chromogenic indicator converting from a colorless state to a colored state, a colored indicator converting from a first color to a distinguishable second color, or a fluorogenic indicator converting from a nonfluorescent state to a fluorescent state. Each of these examples may be visually observable and, in some implementations, the biological indicator may be compared to a “positive control” (i.e., a biological indicator that has not be subjected to a sterilization process and, therefore, contains viable test microorganisms or active enzyme.

In any implementation of the method, the analyzing the biological indicator can comprise placing a portion of the biological indicator into a machine (e.g., a biological indicator auto-reader) that detects the second state of the detection reagent. In some implementations of the method, the machine further quantifies an amount of the second state. In some implementations, the machine quantifies a first amount of the second state at a first time and subsequently quantifies a second amount of the second state at a second time after the first time. A change in the quantity of second state can indicate survival of test microorganisms or active enzyme that had been exposed to the sterilization process.

Because the second portion of the biological indicator extends outside the casing, the biological indicator can be incubated and analyzed using commercial biological indicator auto-readers such as, for example, the 3M™ Attest™ Auto-reader 390 or the 3M™ Attest™ Auto-reader 490, both available from 3M Company (St. Paul, MN). Such auto-readers typically have means to control the incubation temperature of the biological indicators inserted therein and they have an optical system configured to detect (e.g., by fluorescent emission spectra) and determine the quantity of the second state of a detection reagent in the biological indicators.

In any embodiment of the method of the present disclosure, activating the biological indicator can comprise applying a mechanical force against the biological indicator to move a first part of the biological indicator as described hereinabove. Moving the first part relative to the second part (e.g., compressing the cap against the housing) can facilitate sealing the biological indicator and, in some implementations, facilitate releasing the aqueous liquid from the openable (inner) container of the biological indicator. In some implementations of the method, the process challenge device further comprises a plunger, wherein applying the mechanical force against the biological indicator comprises moving the plunger to apply the force against the biological indicator. In some implementations of the method, applying the mechanical force to move a first part of the biological indicator relative to a second part of the biological indicator comprises opening (e.g., by bursting) the openable container holding the aqueous liquid.

In any implementation of the method, the process challenge device further can comprise a chemical integrator device disposed in the cavity of the casing. As disclosed herein, the at least one wall of the casing may be fabricated using an optically-transmissible and, thus, the chemical integrator device can be analyzed (e-g., by visual observation through the at least one wall) without having to remove the chemical integrator device from the process challenge device. In these implementations, the method further comprises without removing the chemical integrator device from the process challenge device, observing the chemical integrator to determine whether the sterilization process achieved at least one parameter associated with an efficacious sterilization process.

### EXAMPLES

### Example 1. Producing a Process Challenge Device Comprising a Plunger-Type Activation Support Member

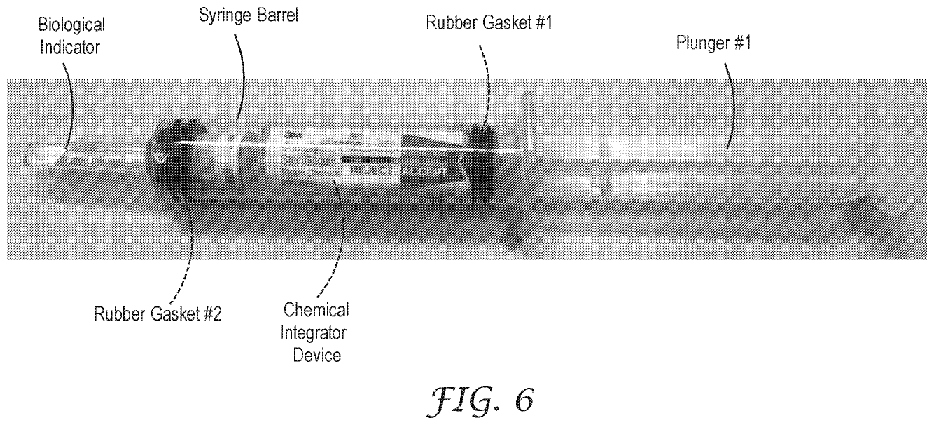

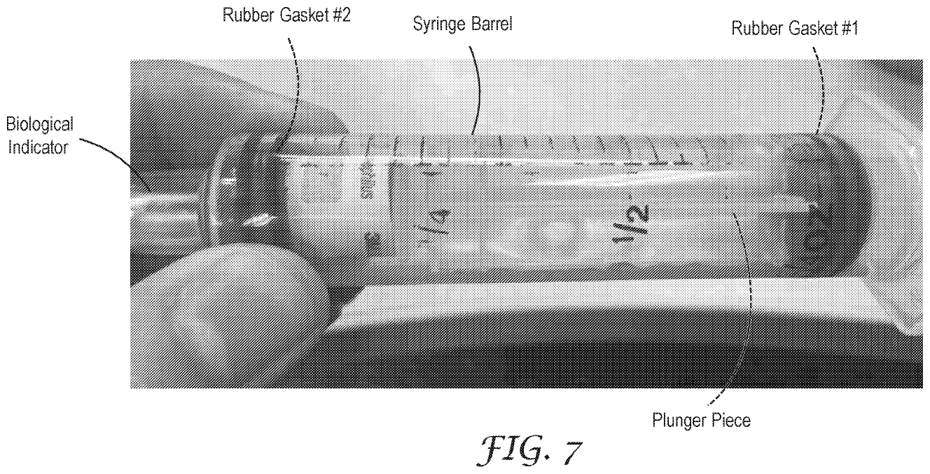

A process challenge device was prepared as shown in FIG. 6. A first 20-ml polypropylene syringe was modified by drilling a hole at the Luer-lock end of the syringe. This hole removed the Luer-lock port and was just large enough in diameter enough to fit around the polycarbonate housing of a commercial biological indicator (3M ATTEST™ Part No. 1295 Rapid Readout Biological Indicator; 3M Company; St, Paul, MN). An additional rubber gasket (rubber gasket #2) taken from the plunger of a second 20-mL syringe was cut to provide a hole so that it could be slipped around the cap of a biological indicator cap as shown in FIG. 6. This gasket prevented leakage of the sterilant gas into the process challenge device and firmly held the biological indicator in place. The plunger of the second syringe (from which gasket #2 was removed) cut in order to have a plunger piece that fits inside of the cavity of the device (see FIG. 7). The plunger piece was positioned to be used for activation of the biological indicator. A STERIGAGE chemical indicator (Part No. 1243R, 3M Company, St. Paul, MN) was then placed inside of the cavity and the rubber gasket (rubber gasket #1) of the plunger (plunger #1) was inserted into the cavity. Finally a small hole (˜1 mm diameter, not shown in FIG. 6 or FIG. 7) was drilled into the wall of the casing (syringe barrel) to allow the steam to enter the cavity and contact the open end of the biological indicator and to contact the chemical integrator device disposed in the cavity.

### Example 2. The Use of a Process Challenge Device Comprising a Plunger-Type Activation Support Member to Assess the Efficacy of a Steam Sterilization Process

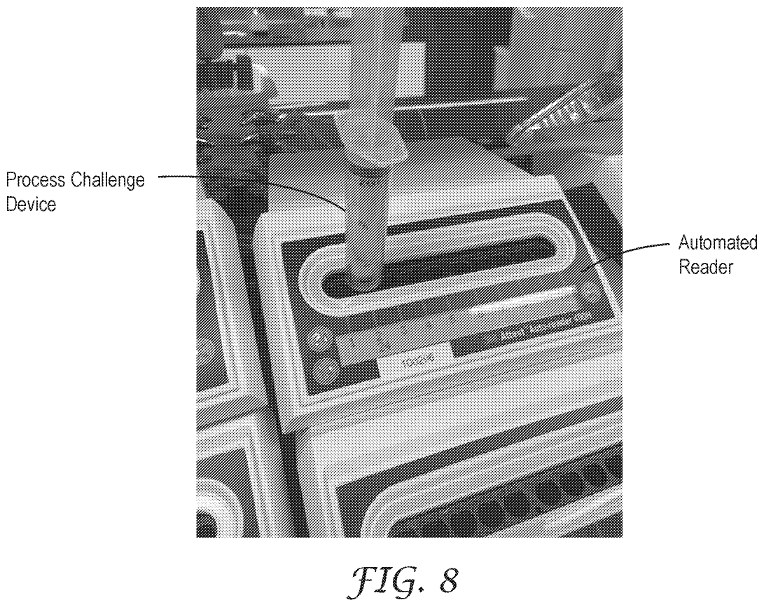

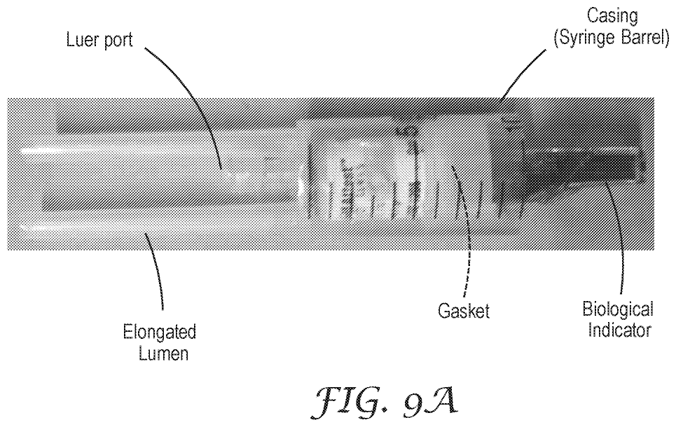

Process challenge devices can be prepared according to Example 1. The devices can be placed into an automated sterilizer (AMSCO Lab 110 steam sterilizer available from Steris Life Sciences: Mentor, OH)) that is programmed to operate a 132° C., 2-pulse dynamic air-removal steam sterilization cycle for 2 minutes at sterilization temperature and a 4-pulse 132° C. cycle for 4 minutes at sterilization temperature. In addition, an AAMI 16-towel pack can be prepared with the same lot of biological indicators and STERIGAGE 1243R chemical integrators can be placed in this same sterilization cycle with a stand-alone biological indicator and chemical integrator. Following the sterilization cycle, the process challenge devices can be removed from the sterilizer and analyzed (i.e., the chemical integrators can be visually observed to see whether the result is “Accept” or “Reject” and the biological indicators can be activated, incubated and analyzed to determine whether the spores were inactivated. FIG. 8 shows that the biological indicators of the process challenge devices of Example 1 can be analyzed using an automated reader (3M ATTEST Auto-reader 4901, available from 3M Company; St. Paul, MN).

### Example 3. Producing a Process Challenge Device Comprising an Elongated Lumen

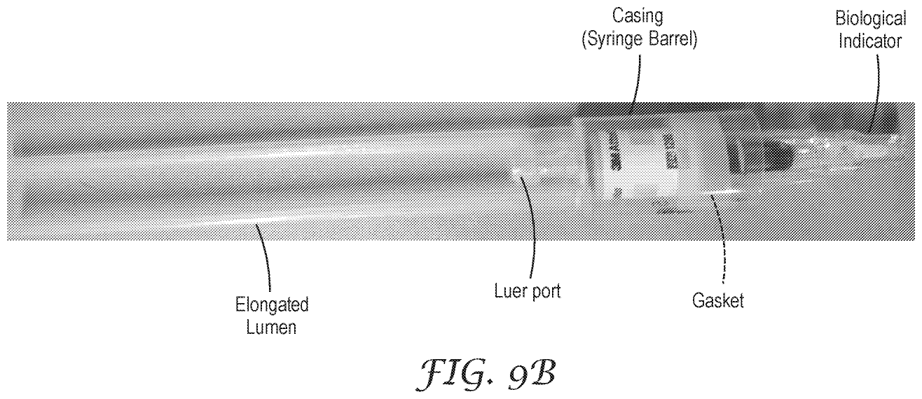

Process challenge device was prepared as shown in FIG. 6. 20-ml polypropylene syringes were cut approximately in half and the dispensing end of the syringes were modified by drilling two holes (approximately 2 mm diameter each); one hole on each side of the Luer-lock port. The Luer-lock ports was scaled wit an adhesive. A piece of silicone tubing was slipped around the middle portion of commercial biological indicators (3M ATTEST™ Part No. 1295 Super Rapid Readout Biological Indicator; 3M Company; St, Paul, MN). The biological indicators with the tubing around them were slipped inside the cut end of the barrel of the modified syringes, as shown in FIGS. 9A and 9B. Pieces (approximately 40 mm long) of silicone tubing (1 mm inner diameter×2 min outer diameter) were inserted into each hole adjacent the Luer port to complete the assembly of the process challenge devices, as shown in FIG. 9A. Alternatively, pieces (approximately 100 mm long) of silicone tubing (1 mm inner diameter×2 mm outer diameter) were inserted into each hole adjacent the Luer port to complete the assembly of the process challenge devices, as shown in FIG. 9B.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. Various features and aspects of the present disclosure are set forth in the following claims.

1. A method of assessing efficacy of a sterilization process, the method comprising:
providing a sterilant gas to an opening of a casing of a process challenge device;
wherein the casing forms a cavity, the casing being is operatively connected to, a self-contained biological indicator;
wherein the biological indicator comprises:
a housing that forms an interior space;
a plurality of viable test microorganisms or an active enzyme disposed in the interior space;
a detection reagent for detecting the viable test microorganisms or the active enzyme, the detection reagent being disposed in the interior space;
a sterilant pathway extending from ambient through the housing to the interior space; and
an aqueous liquid disposed in the interior space, the aqueous liquid being in selective communication with the viable test microorganisms or active enzyme;
wherein the sterilant gas must pass through at least a portion of the casing to contact the test microorganisms, if present, or the active enzyme, if present;
wherein the process challenge device is configured so that the biological indicator can be activated and analyzed without disconnecting the biological indicator from the casing;
wherein providing the sterilant gas to the opening comprises providing the sterilant gas at a predefined temperature for a predefined period of time;
without detaching the biological indicator from the process challenge device, activating the biological indicator by placing the viable test microorganism or active enzyme in contact with the detection reagent; and
without detaching the biological indicator from the process challenge device, analyzing the biological indicator to detect a change in the detection reagent from a first state to a second state;
wherein the efficacy is determined by a presence of a threshold quantity of the second state in the biological indicator after the biological indicator is activated.

2. The method of claim 1, wherein activating the biological indicator comprises applying a mechanical force against the biological indicator to move a first part of the biological indicator relative to a second part of the biological indicator.

3. The method of claim 1, wherein the process challenge device further comprises a plunger, wherein activating the biological indicator comprises applying a mechanical force to the plunger to move a first part of the biological indicator relative to a second part of the biological indicator.

4. The method of claim 2, wherein moving a first part of the biological indicator relative to a second part of the biological indicator comprises opening the openable container.

5. The method of claim 1, further comprising the step of:
before providing the sterilant gas, positioning the process challenge device in a sterilization chamber of an automated sterilizer; wherein providing the sterilant gas to the opening comprises operating the automated sterilizer to provide the sterilant gas into the sterilization chamber.

6. The method of claim 1, wherein analyzing the biological indicator comprises visually observing the biological indicator to detect the change.

7. The method of claim 1, wherein analyzing the biological indicator comprises placing a portion of the biological indicator into a machine that detects the second state of the detection reagent.

8. The method of claim 7, wherein the machine further quantifies an amount of the second state.

9. The method of claim 1, wherein the process challenge device comprises a chemical integrator device disposed in the cavity, wherein the method further comprises observing the chemical integrator device to determine whether the sterilization process achieved at least one parameter associated with an efficacious sterilization process.

10. The method of claim 9, wherein the chemical integrator device is observed without opening the process challenge device and without removing the chemical integrator device from the process challenge device.

11. A process challenge device for verifying efficacy of a sterilization process, the device comprising:
a casing formed of a sterilant vapor-impervious material, the casing forming a cavity with at least one opening that places the cavity in vapor communication with an ambient environment in which the device is disposed; and
a self-contained biological indicator operatively connected to the casing, the biological indicator comprising:
a housing comprising at least one wall, the housing having an open end and an interior space;
a plurality of viable test microorganisms or an active enzyme, the microorganisms or active enzyme being disposed in the interior space of the housing;
a sterilant pathway extending from ambient through the housing to the test microorganisms or the active enzyme;
a detection reagent for detecting the viable test microorganisms or the active enzyme, the detection reagent being disposed in the interior space;
wherein the detection reagent is convertible by the test microorganisms or the active enzyme from a first state to a second state that is distinguishable from the first state;
a fluid pathway extending through the housing to the interior;
an aqueous liquid disposed in an openable container, the aqueous liquid being in selective communication with the viable test microorganisms or active enzyme; and
an activation support member disposed in the cavity of the casing, wherein the activation support member is positioned proximate the open end of the biological indicator,
wherein a first portion of the self-contained biological indicator is disposed in the cavity of the casing and a second portion of the self-contained biological indicator is disposed outside the casing;
wherein the first portion of the biological indicator comprises the open end;
wherein the second portion of the biological indicator comprises a part of the at least one wall through which the aqueous liquid and detection reagent can be observed, and
wherein the activation support member has a first end moveably disposed in the cavity of the casing, wherein the activation support member has a second end disposed outside the casing, and wherein the first end can be moved into contact with the biological indicator.

12-13. (canceled)

14. The process challenge device of claim 11, wherein the casing comprises at least an elongated lumen structure, wherein the at least one opening is disposed in the lumen structure.

15. The process challenge device of claim 11, wherein the biological indicator further comprises a cap, wherein the cap is slidably attached to the housing so that, when the cap moved from a first position to a second position, it closes the open end of the housing.

16. The process challenge device of claim 15 further comprising means for opening the openable container, wherein the openable container is a frangible container, wherein when the cap moved from the first position to the second position, the means for opening the openable container causes the frangible container to open.
